(19) United States
(12) Patent Application Publication
(10) Pub. No.: US 2021/0393678 A1
(43) Pub. Date: Dec. 23, 2021

(54) PERSONALIZED FOOD PRODUCTS FOR ENSURING ADEQUATE IRON INTAKE

(71) Applicant: MY-OR DIAGNOSTICS LTD., Zilkhron Yaakov (IL)

(72) Inventors: Ron KOHN, Kefar Uriya (IL); Michael BRANDWEIN, Elazar (IL); Ariel KATZ, Zikhron Yaakov (IL)

(21) Appl. No.: 17/289,652

(22) PCT Filed: Oct. 31, 2019
(86) PCT No.: PCT/IL2019/051186

§ 371 Date: Apr. 28, 2021

Related U.S. Application Data
(60) Provisional application No. 62/753,110, filed on Oct. 31, 2018.

(51) Int. Cl. A61K 33/26; A61B 5/145; A23L 33/16; A23L 33/15; A61K 45/06

(52) U.S. Cl. CPC A61K 33/26; A61B 5/145; A61K 45/06; A23L 33/15; A23L 33/16

(57) ABSTRACT

The present invention relates to methods for treating iron deficiency in a subject comprising noninvasively determining the hemoglobin level in the subject, and providing the subject with an iron supplement accordingly.

### CROSS REFERENCE TO RELATED APPLICATIONS

This application claims the benefit of priority of U.S. Provisional Patent Application No. 62/753,110 titled “PERSONALIZED FOOD PRODUCTS FOR ENSURING ADEQUATE IRON INTAKE”, filed Oct. 31, 2018, the contents of which is incorporated herein by reference in its entirety.

### FIELD OF THE INVENTION

The present invention relates to methods for treating iron deficiency and/or suppressing or inhibiting developmental disorders in a subject comprising detecting iron deficiency and providing a food product comprising an iron supplement. The present invention also provides related kits and personalized infant nutrition formulations and other food products comprising an iron supplement.

### BACKGROUND

Iron is important in maintaining many of the body's functions, including the production of hemoglobin, the molecule in blood that carries oxygen and is necessary to maintain healthy cells, skin, hair, and nails.

Iron from food is absorbed into the body by the cells that line the gastrointestinal tract, but the body only absorbs a small fraction of the iron ingested. This iron is then released into the blood stream, where the protein transferrin attaches to it and delivers it to the liver. Iron is stored in the liver as ferritin and released as needed to make new red blood cells in the bone marrow. When red blood cells are no longer able to function (after about 120 days in circulation), they are re-absorbed by the spleen. Iron from these old cells can also be recycled by the body.

Iron deficiency is the most common micronutrient deficiency worldwide and the leading cause of anemia in the United States. Iron deficiency is due either to increased need for iron by the body or a decreased absorption or amount of iron taken in. Signs of iron deficiency can include fatigue, decreased work and school performance, difficulty maintaining body temperature, decreased immune function, and glossitis (an inflamed tongue).

Young children are a special risk group for iron deficiency because their rapid growth leads to high iron requirements. Infants and children who are deficient in iron may experience delayed growth and development, including delayed neurodevelopment, and also may be prone to infection. Growth and development of the central nervous system in particular is rapid during the first years of life and iron is critical for this process. The human brain almost triples its weight from birth to 3 years of age and has at that age reached 85% of its adult size. Animal studies have shown that iron is essential for several aspects of brain development, including myelination, monoamine neurotransmitter function, neuronal and glial energy metabolism, and hippocampal dendritogenesis. Case control studies in children have further shown an association between iron deficiency anemia in infancy and long lasting poor cognitive and behavioral performance.

When iron deficiency is diagnosed, the body's iron stores need to be replenished to restore and maintain adequate health. Because individual needs vary, it is important to provide a treatment that ensures adequate intake which is tailored to the individual's unique requirements. Thus, there remains a need in the art for personalized food products, particularly personalized infant formulations, that ensure adequate iron intake to diminish or prevent potential health and developmental consequences.

### SUMMARY

According to a first aspect, there is provided a method for treating iron deficiency in a subject in need thereof, the method comprising the steps of: (i) noninvasively determining the hemoglobin level in the subject and identifying a subject having a hemoglobin level of 10.1-11 g/dL as in need of moderate iron supplementation, a subject having a hemoglobin level of 10 g/dL at most as in need of immoderate iron supplementation, and a subject having a hemoglobin level of at least 11.1 g/dL as in no need of iron supplementation ; and (ii) administering an iron supplement comprising 3-5 mg of iron to the subject determined as in need of moderate iron supplementation and iron supplement comprising 5-7.5 mg of iron to the subject determined as in need of immoderate iron supplementation, thereby treating iron deficiency in the subject.

A method for determining a subject is at increased risk of developing iron deficiency, the method comprising the steps of: (i) noninvasively detecting hemoglobin level in a subject; and (ii) determining a subject having a hemoglobin level of at least 11.1 g/dL has a low risk of developing iron deficiency, a subject having a hemoglobin level of 10.1-11 g/dL has a moderate risk of developing iron deficiency, and a subject having a hemoglobin level of 5-10 g/dL has a high risk of developing iron deficiency, thereby determining iron deficiency in the subject.

In some embodiments, the subject is afflicted with or at increased risk of developing a developmental disorder.

In some embodiments, the subject is any one of: a child, an infant, and a neonate.

In some embodiments, the iron supplement is in the form of an iron chelate.

In some embodiments, the iron chelate comprises an EDTA iron chelate or an iron amino acid chelate.

In some embodiments, the iron amino acid chelate is selected from the group consisting of: ferrous bis glycine chelate, ferric tris glycine chelate, ferric glycinate, and ferrous bis glycinate hydrochloride.

In some embodiments, the iron supplement further comprises a cofactor.

In some embodiments, the cofactor comprises ascorbic acid.

In some embodiments, the cofactor comprises vitamin B6, folic acid, vitamin B12, or a combination thereof.

In some embodiments, the iron deficiency is iron-deficiency anemia or non-anemic iron deficiency.

In some embodiments, the determining comprises measuring iron or hemoglobin in a blood or a tissue sample of the subject.

In some embodiments, noninvasively determining is by a spectroscopy-based method that detects hemoglobin levels in an underlying tissue via a cutaneous sensor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION

A method for treating iron deficiency in a subject in need thereof, the method comprising the steps of: (i) noninvasively determining the hemoglobin level in the subject and identifying a subject having a hemoglobin level of 10.1-11 g/dL as in need of moderate iron supplementation, a subject having a hemoglobin level of 10 g/dL at most as in need of immoderate iron supplementation, and a subject having a hemoglobin level of at least 11.1 g/dL as in no need of iron supplementation ; and (ii) administering an iron supplement comprising 3-5 mg of iron to the subject determined as in need of moderate iron supplementation and iron supplement comprising 5-7.5 mg of iron to the subject determined as in need of immoderate iron supplementation, thereby treating iron deficiency in the subject.

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

The terms “subject,” “individual,” and “patient” are used interchangeably herein, and refer to an animal, for example a human, to whom treatment with a composition or formulation or food product in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. In another embodiment, the child is an infant. In one embodiment, the infant is a neonate. In another embodiment, the infant is an infant born prematurely. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, lapine or porcine. In another embodiment, the subject is mammalian.

As used herein, the term “child” encompasses any subject at the age from infancy to puberty. In some embodiments, a child is at least 2 years old, at least 3 years old, at least 4 years old, at least 5 years old, at least 6 years old, at least 7 years old, at least 8 years old, at least 9 years old, at least 10 years old, at least 11 years old, or at least 12 years old, or any value and range therebetween. Each possibility represents a separate embodiment of the invention. In some embodiments, a child is 1-6 years old, 2-9 years old, 4-11 years old, 6-13 years old, 3-10 years old, 7-12 years old, or 9-11 years old. Each possibility represents a separate embodiment of the invention.

As used herein, the term “infant” encompasses any subject at the age of at least 1 months old, at least 2 months old, at least 4 months old, at least 6 months old, at least 10 months old, at least 12 months old, at least 15 months old, at least 19 months old, at least 22 months old, and 24 months old at most, or any value an range therebetween. Each possibility represents a separate embodiment of the invention. in some embodiments, an infant is 1 to 10 months old, 2 to 15 months old, 3 to 20 months old, 1 to 8 months old, 5 to 24 months old, 7 to 12 months old, 14 to 18 months old, 2 to 22 months old, or 4 to 18 months old. Each possibility represents a separate embodiment of the invention.

As used herein, a “neonate” is 1 day old at most, 2 days old at most, 5 days old at most, 7 days old at most, 12 days old at most, 15 days old at most, 19 days old at most, 22 days old at most, 25 days old at most, or 28 days old at most, or any value and range therebetween. Each possibility represents a separate embodiment of the invention. In some embodiments, a neonate is 1 to 15 days old, 2 to 20 days old, 5 to 17 days old, 7 to 21 days old, or 14 to 28 days old. Each possibility represents a separate embodiment of the invention.

The terms “neonate” and “newborn” are interchangeable.

Conditions and disorders in a subject for which a particular drug, compound, composition, formulation, food product, dietary supplement (or combination thereof) is referred to herein to be “indicated” are not restricted to conditions and disorders for which that drug or compound or composition or formulation or food product or supplement has been expressly approved by a regulatory authority, but also include other conditions and disorders known or reasonably believed by a physician or other health or nutritional practitioner to be amenable to treatment with that drug or compound or composition or formulation or food product or supplement or combination thereof.

In one embodiment, the iron supplement is a food product. In some embodiments, the iron supplement is provided as a part of a food product. In some embodiments, the iron supplement comprises iron drops.

In one embodiment, the iron supplement comprises an iron that has improved absorption compared to an iron salt. In another embodiment, the iron supplement comprises an iron that has improved bioavailability compared to an iron salt. In another embodiment, the iron supplement comprises an iron that is resistant to changes in pH, which keeps iron in a soluble form. In another embodiment, the iron supplement comprises an iron that is resistant to changes in pH, which protects the iron from dietary inhibitors. In one embodiment, the iron supplement comprises or is in the form of an iron chelate. In one embodiment, the iron chelate comprises one or more ferrous iron salts, ferric iron salts, or a combination thereof. In another embodiment, the ferrous iron salt comprises ferrous fumarate, ferrous gluconate, ferrous sulfate, ferrous lactate, ferrous glycine sulfate, or ferrous succinate. In another embodiment, the ferric iron salt comprises ferric ammonium citrate. In another embodiment, the iron chelate comprises an EDTA iron chelate or an iron amino acid chelate. In another embodiment, the iron amino acid chelate comprises ferrous bis glycine chelate, ferric tris glycine chelate, ferric glycinate, ferrous bis glycinate hydrochloride, or any a combination thereof.

In one embodiment, a food product comprises a personalized infant nutrition formulation.

In another embodiment, the dosage of iron within the iron supplement comprises at least 10 mg per dose, at least 25 mg per dose, at least 50 mg per dose, at least 100 mg per dose, at least 200 mg per dosage, at least 300 mg per dosage, or at least 400 mg iron per dosage, or any value and range therebetween. Each possibility represents a separate embodiment of the invention. In another embodiment, the dosage of iron within the iron supplement comprises 10 to 500 mg per dose, 15 to 250 mg per dose, 50 to 450 mg per dose, 100 to 400 mg per dose, 150 to 350 mg per dose. Each possibility represents a separate embodiment of the invention.

In another embodiment, the concentration of iron in the iron supplement ranges from 1 to 25 mg/L. In another embodiment, the concentration of iron in the iron supplement ranges from 5 to 20 mg/L. In another embodiment, the concentration of iron in the iron supplement ranges from 10 to 20 mg/L. In another embodiment, the concentration of iron in the iron supplement ranges from 4 to 17 mg/L. In another embodiment, the concentration of iron in the iron supplement ranges from 3 to 15 mg/L. In another embodiment, the concentration of iron in the iron supplement ranges from 8 to 19 mg/L. In another embodiment, the concentration of iron in the iron supplement ranges from 4 to 12 mg/L. In another embodiment, the concentration of iron in the iron supplement ranges from 2.3 to 12.7 mg/L.

In one embodiment, a method for treating iron deficiency in a subject, comprises the steps of: a) detecting iron deficiency in the subject; b) administering to the subject a an iron supplement, wherein if the iron deficiency in the subject is not severe, the amount of the iron in the iron supplement is low and wherein if the iron deficiency in the subject is severe, the amount of iron in the iron supplement is high.

In one embodiment, a method for suppressing or inhibiting one or more developmental disorders in a subject, comprises the steps of: a) detecting iron deficiency in the subject; b) administering to the subject an iron supplement, wherein if the iron deficiency in the subject is not severe, the amount of the iron in the iron supplement is low and wherein if the iron deficiency in the subject is severe, the amount of the iron in the iron supplement is high, wherein the subject is a child.

As used herein, “severe iron deficiency” encompasses a subject being in need of an immoderate iron supplementation. As used herein, “not severe iron deficiency” encompasses a subject being in need of a moderate iron supplementation.

In one embodiment, a food product is selected from: a snack bar, rice, a cereal, milk, a milk product, a nutrition formula, bread, a snack or a candy. In another embodiment, the food product is a personalized infant nutrition formulation.

In one embodiment, iron deficiency is iron-deficiency anemia or non-anemic iron deficiency.

In one embodiment, the step of detecting iron deficiency comprises a noninvasive iron detection method. In another embodiment, the step of detecting iron deficiency comprises a noninvasive hemoglobin detection method. In another embodiment, the noninvasive detection method comprises a spectroscopy-based method that detects iron or hemoglobin levels in underlying tissue via a cutaneous sensor.

As used herein, the term “underlying tissue” refers to a tissue located under the skin or cutaneous layer. In some embodiments, the underlying tissue is a blood vessel.

In some embodiments, the blood vessel is a capillary, a venule, arteriole, artery, vena, sinus, or any combination thereof.

In some embodiments, the herein disclosed method covers any spectroscopy-based method which relies on the differential light absorption of oxygenated hemoglobin (HbO₂) and deoxygenated hemoglobin (Hb), to compute their respective concentrations in the arterial blood. In some embodiments, the spectroscopy-based method comprises occlusion spectroscopy.

In some embodiments, differential absorption can be measured at the red and infrared wavelengths of the sensor. The relationship between arterial oxygen saturation and hemoglobin concentration can be expressed as:

SpₐO₂=100; C_{HbO2}/(C_{Hb}+C_{HbO2}) (1)

That is, arterial oxygen saturation is the percentage concentration of oxygenated hemoglobin compared to the total concentration of oxygenated hemoglobin and deoxygenated hemoglobin in the arterial blood. SpₐO₂ is actually a measure of the partial oxygen saturation of the hemoglobin because other hemoglobin derivatives, such as COHb and MetHb, are not taken into consideration.

In addition to the differential absorption of hemoglobin derivatives, the spectroscopy-based method can rely on the pulsatile nature of arterial blood to differentiate hemoglobin absorption from absorption of other constituents in the surrounding tissues. Light absorption between systole and diastole varies due to the blood volume change from the inflow and outflow of arterial blood at a peripheral tissue site, e.g., skin.

Non-limiting examples for devices adequate for a spectroscopy-based method according to principals described hereinabove, include but are not limited to the such as described in U.S. Pat. Nos. 6,898,452, and 9,913,602.

In one embodiment, the noninvasive iron detection method further comprises microbiome detection on the skin of the subject. In one embodiment, the microbiome detection comprises the steps of: a) collecting a biological sample from skin of the subject; b) analyzing the microbiome in the sample, wherein differences in the combination of bacterial strains present in the sample as compared to the combination of bacterial strains present in non-iron-deficient subjects and similarities in the combination of bacterial strains present in the sample as compared to the combination of bacterial strains present in iron-deficient subjects are diagnostic of iron deficiency in the subject.

In one embodiment, the dose of iron to be supplemented is personalized in an age-dependent manner. In one embodiment, the iron dose ranges from 0.9-1.3 mg/kg/day for a subject that is 6-12 months old. In one embodiment, the iron dose ranges from 0.5-0.8 mg/kg/day for a subject that is 1-3 years old.

In another embodiment, provided herein is a kit for detecting and treating iron deficiency in a subject, comprising: a) a non-invasive iron detector; b) one or more iron supplements ; and c) instructions for use, wherein if the kit comprises more than one iron supplement, each which comprises iron in a different concentration.

In another embodiment, the one or more iron supplements are provided in trial-sized containers.

In another embodiment, the kit comprises three distinct iron supplement comprising a low concentration of iron, a midlevel concentration of iron, and a high concentration of iron, wherein the concentration of iron in each of the iron supplements are within the range approved by the local regulatory authority.

In one embodiment, an iron supplement as described herein is provided in a dosage of 30-60 mg/day elemental iron. In another embodiment, the iron supplement is provided in a dosage of 150-300 mg/day of ferrous sulfate heptahydrate. In another embodiment, the iron supplement is provided in a dosage of 90-180 mg/day of ferrous fumarate. In another embodiment, the iron supplement is provided in a dosage of 250-500 mg/day of ferrous gluconate. In another embodiment, the iron supplement is provided in a dosage of 150 mg/day of amino acid iron chelate.

In one embodiment, the iron supplement, iron chelate, or food product comprising the same is selected from a snack bar, rice, cereal, milk, a milk product, a nutrition formula, bread, a chocolate, gummy bears, gummy worms, jellybeans. Any other food or nutritional product or supplement, or liquid, solid, or semisolid formulation or product for oral consumption and suitable for use in accordance with the formulations and methods described herein can be employed.

### Iron Deficiency

As used herein, the term “Iron deficiency” generally includes a decrease of the total iron content in the body, as would be apparent to one of ordinary skill in the art. Many different measures of iron status are available, and different measures are useful at different stages of iron depletion. Measures of serum ferritin can be used to identify iron depletion at an early stage. A reduced rate of delivery of stored and absorbed iron to meet cellular iron requirements represents a more advanced stage of iron depletion, which is associated with reduced serum iron, reticulocyte hemoglobin, and percentage transferrin saturation and with higher total iron binding capacity, red cell zinc protoporphyrin, and serum transferrin receptor concentration. The last stage of iron deficiency, characterized by iron-deficiency anemia, occurs when blood hemoglobin concentrations, hematocrit (the proportion of red blood cells in blood by volume), mean corpuscular volume, and mean cell hemoglobin are low. Hemoglobin and hematocrit tests are the most commonly used measures to screen patients for iron deficiency, even though they are neither sensitive nor specific. Hemoglobin concentrations lower than 13 g/dL in men and 12 g/dL in women indicate the presence of iron-deficiency anemia. Normal hematocrit values, which are generally three times higher than hemoglobin levels, are approximately 41% to 50% in males and 36% to 44% in females. (National Institutes of Health, Office of Dietary Supplements).

In some embodiments, hemoglobin level in the blood reflects the iron level in the blood. In some embodiments, hemoglobin level in the blood correlates or comprises the iron level in the blood.

The terms “hemoglobin level” and “iron level” are used herein interchangeably.

As used herein, a subject having a hemoglobin level of 11 g/dL at most is in need of iron supplementation. In some embodiments, a subject having a hemoglobin level of 10 g/dL at most is administered with an iron dose of at least 5 mg per kg body weight per day, according to the present method. In some embodiments, a subject having a hemoglobin level of 10.1 to 11 g/dL is administered with an iron dose of 3-5 mg per kg body weight per day, according to the present method.

In some embodiments, a subject in need of moderate iron supplementation has a hemoglobin level of 10.1-11 g/dL. In some embodiments, a subject in need of immoderate iron supplementation has a hemoglobin level of 5-10 g/dL. In some embodiments, a subject having a hemoglobin level of 11.1-13 g/dL is of no need of iron supplementation.

Iron deficiency can be diagnosed by any suitable method known in the art, including, without limitation, testing serum iron, transferrin, total iron-binding capacity (TIBC), unsaturated iron-binding capacity (UIBC), transferring saturation, serum ferritin, hemoglobin, hematocrit, mean cellular volume (MCV), or a combination thereof. Normal hemoglobin levels vary among individuals, based on sex, age, and other factors and recognition of such normal levels, and thus abnormal levels, is within the knowledge of one of ordinary skill in the art.

In the early stage of iron deficiency, physical effects might not be observed, and the amount of iron stored may be significantly depleted before any signs or symptoms of iron deficiency develop.

Insufficient levels of circulating and stored iron may eventually become severe and can lead to iron-deficiency anemia (characterized by decreased hemoglobin and hematocrit, and smaller and paler red cells). In iron-deficiency anemia, iron stores are exhausted, hematocrit and levels of hemoglobin decline, and the resulting microcytic, hypochromic anemia is characterized by small red blood cells with low hemoglobin concentrations. Iron deficiency anemia can be defined as a hemoglobin level that is lower than two standard deviations from the mean distribution in a healthy population of the same gender and age living at the same altitude. At sea level, hemoglobin concentrations lower than 11 to 12 g/dL in children younger than 12, 12 g/dL in adolescents and women, and 13 g/dL in men indicate the presence of iron-deficiency anemia.

If a person is otherwise healthy and anemia develops over a long period of time, symptoms seldom appear before the hemoglobin in the blood drops below the lower limit of normal. However, as the iron deficiency progresses from non-severe to severe, for example, symptoms of anemia eventually begin to appear. The most common such symptoms include fatigue, weakness, dizziness, headaches and pale skin.

The functional deficits associated with anemia include gastrointestinal disturbances and impaired cognitive function, immune function, exercise or work performance, and body temperature regulation. In infants and children, iron-deficiency anemia can result in psychomotor and cognitive abnormalities that, without treatment, can lead to learning difficulties. Some evidence indicates that the effects of deficiencies early in life persist through adulthood.

Groups at risk of iron inadequacy include pregnant women, infants and young children, women with heavy menstrual bleeding, frequent blood donors, people with cancer, people with gastrointestinal disorders or who have had gastrointestinal surgery, and people with heart failure.

In some embodiments, the subject is afflicted with a developmental disorder. In some embodiments, the subject is at increased risk of developing a developmental disorder. In some embodiments, increased risk is in comparison to a control. In some embodiments, a control comprises a subject having normal hemoglobin levels (e.g., blood levels). In some embodiments, normal hemoglobin levels are at least 11.1 g/dL, at least 11.5 g/dL, at least 12 g/dL, at least 12.5 g/dL, or at least 13 g/dL, or any value and range therebetween. Each possibility represents a separate embodiment of the invention. In some embodiments, normal hemoglobin levels are 11.1-15 g/dL, 11.5-13.5 g/dL, 12.4-13.9 g/dL, or 13.1 to 14.7 g/dL. Each possibility represents a separate embodiment of the invention.

Treatment of any one of these groups or individuals, as well as any others at any stage of iron deficiency, is contemplated within the scope of the present invention.

### Iron Deficiency Detection

Methods of detecting iron and hemoglobin and thus, iron deficiency, in a subject are known in the art and readily available and can include invasive, minimally invasive, or noninvasive methods. Any such methods suitable for use in accordance with the methods described herein can be employed. In an embodiment, iron or hemoglobin detection comprises obtaining a blood sample from the subject. In another embodiment, iron or hemoglobin detection comprises obtaining a tissue sample from the subject. In another embodiment, iron or hemoglobin detection is achieved without the need for a blood or tissue sample.

### Skin Microbiome Analysis

In some embodiments, the noninvasive iron detection comprises microbiome detection on the skin of the subject. In some embodiments, methods are provided which comprise, inter alia, collecting a biological sample from the skin of a subject and analyzing the microbiome in the sample collected from the skin. Modern techniques for skin microbial analysis are known and within the understanding of the ordinarily skilled artisan and specific methods and techniques can be employed and adjusted to best suit the aims of the study (See e.g. Weyrich, et al.).

In general, several procedural practices should be considered before the study begins, including avoiding contamination by environmental DNA, storage in warm conditions, and the exposure of the samples to researchers and clinicians. Further, precautions should be taken to ensure a sterile technique is utilized, and that bacterial DNA sequences (not only live bacterial organisms) are not introduced into the sample from sampling equipment, lab reagents, clinicians, etc. (Id.).

To obtain samples, microorganisms from the skin can be collected by any suitable method known in the art, including, without limitation, swabbing, scraping or collecting biopsies using sterile techniques. Skin samples can be collected from any suitable location, including, without limitation, the nare, axillary vault, antecubital fossa, interdigital webspace, inguinal crease, gluteal crease, popliteal fossa, plantar heel, umbilicus, or a combination thereof. Appropriate and effective sample storage conditions should also be employed. If sterile sample collection is combined with effective storage conditions, an accurate representation of the skin microbiome should be maintained prior to DNA extraction and analysis. Once the samples are obtained and properly stored, DNA extractions can then be performed. Several different methods have been developed for the extraction of skin microbiome samples, including the REPLI-g Midi kit (Qiagen, Limberg, The Netherlands), Qiagen DNA Extraction Kit (Qiagen), and DNeasy DNA Extraction kit (Qiagen). In an effort to obtain the most accurate representation of the microbial diversity, studies have also explored different kit and non-kit based extraction methods, such as disruption of bacterial cell walls (e.g., bead beating or enzymatic lysis) (Id.).

After DNA extraction, the specific target species or classes of microorganisms need to be identified to determine the most appropriate sequencing strategy. For example, bacterial communities can be assessed by amplifying a variable region of the conserved 16S ribosomal RNA gene (16S), while fungal species can be targeted by applying 18S ribosomal RNA gene or the internal transcribed spacer (Id.).

While culturing methods can be used in detecting bacterial strains in accordance with embodiments described herein, targeted sequencing approaches do not require any culturing methods and hundreds of samples can be analyzed on a single sequencing run, providing an efficient and cost-effective means to examining microbial communities. Alternatively, shotgun sequencing can be performed, which will identify a subset of random DNA sequences from the sample. In either approach, sequencing technologies should also be taken into account. While Roche 454 or Illumina MiSeqs can provide adequate sequencing coverage or depth for targeted amplicon sequencing, deeper coverage attainable through Illumina HiSeq or Pacific Biosciences technologies may be better suited for shotgun sequencing (Id.).

### 16S Data Processing

The 16S sequence data can processed in accordance with any suitable techniques known to one of skill in the art, including as described in McDonald et al., (2018). In one embodiment, processing can use a sequence variant method, such as Deblur v1.0.2, trimming to 125 nucleotides (nt) (otherwise default or other suitable parameters), to maximize the specificity of 16S data. Following processing by Deblur, previously recognized bloom sequences can be removed. The Deblur sOTUs can be inserted into the Greengenes 13_8 (19) 99% reference tree using SEPP. SEPP uses the simultaneous alignment and tree estimation strategy described by Liu, et al., (2009) to identify reasonable placements for sequence fragments within an existing phylogeny and alignment. Taxonomy can be assigned using an implementation of the RDP classifier as implemented in QIIME2. (McDonald, et al.).

Principal coordinates analysis can be undertaken in accordance with any suitable techniques known to one of skill in the art. In one embodiment, a distance matrix can be constructed using, for example, without limitation, the Bray Curtis dissimilarity index. In one embodiment, principal coordinates analyses can be implemented using, for example, without limitation, EMPeror software.

Bray Curtis dissimilarity is used to quantify the differences in species populations between two different sites, and can be calculated with the following formula:

$$B {C}_{i j} = 1 - \frac{2 {C}_{i j}}{{S}_{i} + {S}_{j}}$$

where i & j are the two sites, Si is the total number of specimens counted on site i, Sj is the total number of specimens counted on site j, Cij is the sum of only the lesser counts for each species found in both sites.

### Bacterial Strains

In some embodiments, a vast number of bacterial strains potentially present in the skin microbiome can be detected and analyzed, and information obtained therefrom used in accordance with the methods and various techniques described herein.

In one embodiment, bacterial strains detected in a skin sample obtained in accordance with methods described herein are selected from: Acidaminococcus, Acinetobacter, Actinomyces, Actinobacteria, Aerococcus, Anaerococcus, Arcanobacterium, Atopobium, Atopobium vaginae, Bacteroides ovatus, Bacteroides uniformis, Brevibacillus, Brevibacterium paucivorans, Campylobacter ureolyticus, Cellvibrio, Citrullus lanatus, Coprococcus, Corallococcus exiguus, Corynebacterium, Corynebacterium kroppenstedtii, Cyanobacteria, Dermabacter, Dialister, Enhydrobacter, Enterobacteriaceae, Facklamia, Faecalibacterium prausnitzii, Finegoldia, Firmicutes, Flavobacterium, Gallicola, Gardnerella, Gemella, GW-34, Haemophilus, Lachnospiraceae, Micrococcaceae, Moryella indoligenes, Parabacteroides, Parvimonas, Pasteurelllaceae, Peptococcus, Peptoniphilus, ph2, Porphyromonas, Prevotella, Proteobacteria, Providencia, Pseudoclavibacter bifida, Pseudomonas, Pseudonocardia, Rhodobacter, Rickettsiella, Roseburia, Roseburia faecis, Ruminococcus, Scardovia, Shuttleworthia, Sneathia, Sneathia, Sphingomonas, Staphylococcaceae, Streptococcus anginosus, Streptococcus, Streptococcus sobrinus, Thermoactinomyces, Wautersiella, Alicyclobacilus, Bacillus cereus, Blastomonas, Chryseobacterium, Comamonas, Erythromicrobium, Fusobacterium, Gardnerella, Gordonia, Gyrocarpus, Haemophilus parainfluenzae, Lupinus, Paracoccus, Phenylobacterium, Prevotella, Prevotella copri, Sphingobium yanoikuae, Stenotrophomonas, Streptococcus, Streptococcus infantis, Vagococcus, Veillonellaceae, or any combination thereof.

### Iron Supplements

In some embodiments, provided herein are, inter alia, infant nutrition formulations and other food products comprising an iron supplement, and methods and kits comprising the use of such supplements. Various forms of iron for inclusion in iron supplements are known and readily available and any such forms suitable for use in accordance with the formulations, products, and methods described herein to achieve desired results can be employed, alone or in combination. In one embodiment, the iron in a supplement described herein comprises iron in the form of an iron chelate. In another embodiment, the iron chelate comprises one or more ferrous iron salts, ferric iron salts, or a combination thereof. In another embodiment, the ferrous iron salt comprises or is selected from: ferrous fumarate, ferrous gluconate, ferrous sulfate, ferrous lactate, ferrous glycine sulfate, ferrous succinate, or any combination thereof. In another embodiment, the ferric iron salt comprises ferric ammonium citrate. In another embodiment, the iron chelate comprises an EDTA iron chelate or an iron amino acid chelate. In another embodiment, the iron amino acid chelate comprises ferrous bis glycine chelate, ferric tris glycine chelate, ferric glycinate, ferrous bis glycinate hydrochloride, or any combination thereof. In an embodiment, the iron chelate comprises Ferrochel®. Any other suitable form of supplemental iron also can be employed, including, without limitation, ferric sulfate, ferric citrate, ferrous ascorbate, heme iron polypeptides, carbonyl iron, and polysaccharide-iron complexes, or a combination thereof.

In another embodiment, a food product as described herein, alone and for use in the methods as described herein may comprise a co-factor so as to normalize hemoglobin levels. In one embodiment, the cofactor comprises vitamin B6, folic acid, vitamin B12, vitamin C (ascorbic acid), or any combination thereof. In one embodiment, the food product comprises an iron supplement and a cofactor as described hereinabove. In another embodiment, the food product comprises vitamin B6, folic acid, vitamin B12, vitamin C (ascorbic acid), or any combination thereof, devoid of an iron supplement.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, iron deficiency-related disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the disease severity, delaying the disease onset, reducing the disease cumulative incidence, or any combination thereof.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

As used herein, the terms “component,” “composition,” “formulation”, “composition of compounds,” “compound,” “drug,” “pharmacologically active agent,” “active agent,” “therapeutic,” “therapy,” “treatment,” “medicament,” or “food product” are used interchangeably herein, as context dictates, to refer to a compound or compounds or composition of matter which, when administered to a subject (human or animal) induces a desired pharmacological and/or physiologic effect by local and/or systemic action. A personalized formulation refers to a product or use of the product in a regimen tailored or individualized to meet specific needs identified or contemplated in the subject.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

When values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

All patents, patent applications, and scientific publications cited herein are hereby incorporated by reference in their entirety.

The following examples are presented in order to more fully illustrate the preferred embodiments of the invention. It should in no way be construed, however, as limiting the broad scope of the invention.

### EXAMPLES

### Example 1

Clinical studies with Albion's flagship product, Ferrochel® (ferrous bis glycinate—iron amino acid chelate) have shown Ferrochel® improved hemoglobin and ferritin indices at lower dosages than ferrous sulfate or ferrous ascorbate; lower dosages mean fewer side effects and no interactions with other nutrients.

### Example 2

As the general population comprises subpopulations of moderate and immoderate iron deficient subjects, as described herein above, there is a need for personalized iron dosing so as to avoid adverse effects, including toxicity, and under- or over-dosing, etc., which result from a “one treatment fits all” approach.

To examine this, the inventors have recruited a population (2-7 years of age) of immoderate iron deficient subjects having an average hemoglobin level of 10.03 g/dL. Total blood hemoglobin levels were measured noninvasively at baseline (time 0) and following treatment (after 2 weeks) from the index finger of subjects using Masimo rainbow SET SpHb technology. Subjects were instructed to take 1 mL of iron bis-glycinate chelate (15 mg) daily. Following two weeks of treatment, total blood hemoglobin levels were measured as described above, and the results showed hemoglobin level had increased by 11% on average (11.13 g/dL).

Therefore, tailoring iron chelate dose per a specific iron deficiency type subpopulation (e.g., moderate or immoderate) together with a noninvasive monitoring is required, so as to increase hemoglobin level in an effective and safe manner, as shown herein.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-14. (canceled)

15. A method for treating iron deficiency in a subject in need thereof, the method comprising the steps of:
a) noninvasively determining the hemoglobin level in said subject and identifying a subject having a hemoglobin level of 10.1-11 g/dL as in need of moderate iron supplementation, a subject having a hemoglobin level of 10 g/dL at most as in need of immoderate iron supplementation, and a subject having a hemoglobin level of at least 11.1 g/dL as in no need of iron supplementation; and
b) administering an iron supplement comprising 3-5 mg of iron to the subject determined as in need of moderate iron supplementation and iron supplement comprising 5-7.5 mg of iron to the subject determined as in need of immoderate iron supplementation,
thereby treating iron deficiency in the subject.

16. The method of claim 15, wherein said subject is afflicted with or at increased risk of developing a developmental disorder.

17. The method of claim 15, wherein said subject is any one of: a child, an infant, and a neonate.

18. The method of claim 15, wherein said iron supplement is in the form of an iron chelate.

19. The method of claim 18, wherein said iron chelate comprises an EDTA iron chelate or an iron amino acid chelate.

20. The method of claim 19, wherein said iron amino acid chelate is selected from the group consisting of: ferrous bis glycine chelate, ferric tris glycine chelate, ferric glycinate, and ferrous bis glycinate hydrochloride.

21. The method of claim 15, wherein said iron supplement further comprises a cofactor.

22. The method of claim 21, wherein said cofactor comprises ascorbic acid.

23. The method of claim 21, wherein said cofactor comprises vitamin B6, folic acid, vitamin B12, or a combination thereof.

24. The method of claim 15, wherein said iron deficiency is iron-deficiency anemia or non-anemic iron deficiency.

25. The method of claim 15, wherein said determining comprises measuring iron or hemoglobin in a blood or a tissue sample of said subject.

26. The method of claim 15, wherein said noninvasively determining is by a spectroscopy-based method that detects hemoglobin levels in an underlying tissue via a cutaneous sensor.

27. The method of claim 15, wherein said subject is a newborn, an infant, or a child.

28. The method of claim 15, wherein said iron supplement comprises a food product or iron drops.

29. The method of claim 15, wherein said iron supplement is provided as a part of a food product.

30. The method of claim 28, wherein said food product comprises a personalized infant nutrition formulation.

31. The method of claim 28, wherein said food product is selected from the group consisting of: a snack bar, rice, a cereal, milk, a milk product, a nutrition formula, bread, a snack or a candy.
